(19) United States
(12) Patent Application Publication
(10) Pub. No.: US 2020/0290038 A1
(43) Pub. Date: Sep. 17, 2020

(54) PREFILLED CARTRIDGE

(71) Applicants: Didier Falconnet, Vufflens-la-Ville (CH); Lucienne Lagopoulos (Robert-Charrue), Borex (CH)

(72) Inventors: Didier Falconnet, Vufflens-la-Ville (CH); Lucienne Lagopoulos (Robert-Charrue), Borex (CH)

(73) Assignee: MYCARTIS N.V., Zwijnaarde (BE)

(21) Appl. No.: 16/341,787

(22) PCT Filed: Sep. 28, 2017
(86) PCT No.: PCT/EP2017/074659

§ 371 Date: Apr. 12, 2019

(30) Foreign Application Priority Data
Oct. 12, 2016 (EP) 16193581.2

(51) Int. Cl. B01L 3/00; G01N 33/543

(52) U.S. Cl. CPC B01L 3/502707; G01N 33/54366; B01L 2300/16; B01L 2200/16; B01L 2200/12

(57) ABSTRACT

The disclosure pertains to a microfluidic cartridge comprising at least one microchannel and at least a set of functionalized microcarriers, the microcarriers being localized within the microchannel, wherein the functionalized microcarriers are coated with at least a lyoprotectant. The disclosure further pertains to a process of manufacture of a microfluidic cartridge according to the invention, said process comprising: providing a microfluidic cartridge comprising at least one microchannel and at least a set of functionalized microcarriers, preferably in suspension in a buffer solution, the microcarriers being localized within the microchannel; flowing a stabilizing buffer into the at least one microchannel and incubating the functionalized microcarriers with said stabilizing buffer for at least 10 minutes, wherein the stabilizing buffer is a composition comprising a lyoprotectant, preferably wherein the lyoprotectant is chosen from the list consisting of sugars and sugar alcohols and mixtures thereof; and drying the at least one microchannel.

The invention pertains to the field of biological and chemical assays performed using microfluidics. The invention provides improved cartridge to be used in automated microfluidic devices, to perform said biological and chemical assays. The invention more precisely pertains to prefilled cartridges comprising microchannels at least partially filled with microcarriers harboring detection molecules, and methods to prepare such cartridge.

An important problem in the field of life sciences and healthcare is simple and rapid detection of biomarkers in a limited amount of biological sample. Microfluidic devices have provided a breakthrough in this respect, as they enable a very accurate detection with low sample volume requirement.

For instance, platforms using pressure driven microfluidics, such as pressure driven laminar flow, use microfluidic channels, or microchannels, to transport the fluids. The biological or chemical assays are performed using microparticles or microcarriers, which are functionalized with detection molecules, such as antibodies or oligonucleotides, and optionally with labeling means, such as fluorophores. As the technique keeps evolving towards miniaturized tools, cartridges appropriate for use with these platforms have been developed, which typically present with microchannels serving as fluid transportation channels, reaction chamber and detection chamber. The functionalized microcarriers are introduced in the microchannels, and the sample to be tested is flown in the cartridge.

Although such microfluidic cartridges have proven very useful, their implementation still remains delicate and time consuming. Indeed, since they require the use of biological molecules for the detection of the biomarker, the coupling of the detection molecule to the microcarriers need to be realized just prior to their actual use to avoid degradation of the detection molecule. As a consequence, the detection molecules are usually provided separately from the microcarriers and the core of the cartridge, and need to be transported and stored at low temperature or lyophilized prior to being coupled to the microcarriers. This is particularly important when the detection molecule is a protein such as an antibody or an enzyme, which rapidly degrades at room temperature. Then, typically, the detection molecules are grafted on the microcarriers before their use, and the functionalized microcarriers are then introduced in the microchannels of the core of the cartridge. Because of their extremely small dimensions, the microcarriers need to be introduced in the microchannel by some specific process, which may involve intricate manipulation of individual microcarriers. As a result, those steps may take up to several hours. In the meantime, the biological samples to be tested have to be stored in appropriate conditions so as to avoid their degradation.

These technical constraints are not only time consuming, they also require a specific equipment for the appropriate storage of both the detection molecules and the biological samples. Overall, they represent a major impediment that prevents efficient point-of-care testing.

There is thus a need for prefilled cartridge which would already contain in their microchannels the functionalized microcarriers, that is to say the detection molecules attached on the microcarriers. In addition, there is a need to simplify storage conditions of the cartridge and its reagents, and provide cartridges that can be easily transported and stored without refrigeration.

To overcome this issue, some have proposed microfluidic cartridges pre-filled with reagents, i.e. ready-to use microfluidic cartridges having on board reagents. Such technical solution has been described by Chen et at (Current Opinion in Chemical Biology, 10:226-231, 2006), which disclosed microfluidic cartridges preloaded with nanoliter plugs of reagents. In this set up, the cartridge is formed of capillary plugs loaded with reagent. The cartridge may be plugged to the device in a merging junction, so as to be connected to a receiving capillary. Buffers and/or samples may be introduced in the capillary plugs of the cartridge, thereby mixing with the reagents and flowing toward the receiving capillary, wherein the reaction takes place.

However, this technical solution is limited to set-ups that do not require sophisticated manipulations of fluids. In addition, the reagents are present in the cartridge in a liquid form. As a result, specific means such as an impermeable fluorinated carrier fluid may be necessary to prevent the reagents from evaporating. In addition, although this technical solution provides cartridges that are ready to use, it does not solve the issue of transportation and storage at room temperature.

Another technical solution which has been investigated is the use of lyophilized (freeze-dried) reagents. Lyophilisation is a well-known preservation technique by which a dry product is obtained through freezing the product and subsequently sublimating the ice formed in low pressure conditions. Although this technique has been successfully implemented for the extended storage of biological material at room temperature, it is hardly compatible with the technical constraints of microfluidic devices, and cannot readily be implemented in the manufacturing of microfluidic cartridges. In particular, it can hardly be implemented to freeze-dry material within a microchannel, wherein fluids do not behave according to the classical physics of fluids. The technique thus can only be used to prepared freeze-dried reagents that still need to be rehydrated and introduced in the microfluidic device before the assays, but does not enable the manufacture of ready-to use pre-filled cartridge.

Thus, there is a need for improved microfluidic cartridges that would be ready to use as well as transportable and storable in routine conditions, that is to say would have a good shelf life even at room temperature and above.

The invention provides a technical solution to the problem at hand.

### DESCRIPTION OF THE INVENTION

The inventors have designed a microfluidic cartridge comprising microchannels at least partially filled with functionalized microcarriers, which are ready to use and surprisingly stable at a large range of temperature conditions for several days, even weeks. As shown in the experimental part, the microfluidic cartridge of the invention is advantageously stable when stored at room temperature and above, even when the detection molecules used are molecules known to be particularly sensitive to degradation. Without being bound by theory, the lyoprotectant coating is likely to help stabilizing the detection molecules and/or the label which are attached to the surface of the microcarriers.

The invention thus pertains to a microfluidic cartridge comprising at least one microchannel and at least a set of functionalized microcarriers, the functionalized microcarriers being localized within the functionalized microchannel, wherein the functionalized microcarriers are coated with at least a lyoprotectant.

The invention further pertains to a method of manufacture of said microfluidic cartridge, comprising the steps of:
providing a microfluidic cartridge comprising at least one microchannel and at least a set of functionalized microcarriers, the functionalized microcarriers being localized within the microchannel;
flowing a stabilizing buffer into the at least one microchannel and incubating the functionalized microcarriers with said stabilizing buffer for at least 10 minutes; and
drying the at least one microchannel.

### DETAILED DESCRIPTION

The invention pertains to a microfluidic cartridge comprising at least one microchannel and at least a set of functionalized microcarriers, the microcarriers being localized within the microchannel, wherein the microcarriers are coated with at least a lyoprotectant.

By “microfluidic cartridge” it is herein referred to a disposable cartridge appropriate for use in a microfluidic device, preferably pressure driven microfluidic device.

By “microchannel” or “microfluidic channel” it is herein referred to a hollow structure appropriate for the passage of fluids, i.e. an enclosed passage, having sub-millimeter dimensions. Preferably, the at least one microchannel according to the invention has a cross-section microscopic in size, i.e. with the largest dimension (of the cross-section) being typically from 1 to 500 micrometers, preferably 10 to 500 micrometers, more preferably from 20 to 400 micrometers, even more preferably from 30 to 400 micrometers. When referring to the “cross-section”, the cross-section perpendicular to the longitudinal axis is meant. A microchannel typically has, at one end, an entry and, at the other end, an exit, which are openings in the microchannel that e.g. let the fluids enter into the microchannel, respectively leave the microchannel. The entry may be connected to an inlet well, the exit may be connected to an outlet well.

The appropriate dimensions and material of the microchannels may easily he determined by the person skilled in the art according to common knowledge in the field. Microchannels appropriate for the invention have for instance been described in WO 2010/072011.

The microchannel and microcarriers may be designed to facilitate mass transfer of the fluids and/or the microcarriers within the microchannels, so as to guaranty accuracy of the data. Ways to design such microchannel and microcarriers have been described in WO 2010/072011. In an embodiment, the shape and size of the microcarriers relative to the cross-section of the at least one microchannel allows to have, over the entire length of the microchannel, at least two of any of the microcarriers arranged side by side without touching each other and without touching the perimeter of the microchannel when travelling in the longitudinal direction of the microchannel.

The microfluidic cartridge of the invention may comprise one or several microchannels. Preferably, the microfluidic cartridge comprises more than one microchannel.

By “microcarrier” or “microcarriers” it is herein referred to any type of particles microscopic in size, typically with the largest dimension being from 100 nm to 300 micrometers, preferably from 1 μm to 200 μm. The microcarriers of the invention may be made from or comprise any material routinely used in high-throughput screening technology and diagnostics. Non-limiting examples of these materials and shapes are disclosed in WO 2010/072011. In a preferred embodiment, the microcarriers have a disk-like shape with the front face in form of a circle.

Microfabrication techniques to manufacture microchannels and microcarriers are known in the art and have for instance been detailed in techniques that are extensively described in the literature (Fundamentals of microfabrication, Madou M., CRC Press, 2002, and Fundamentals and Applications of Microfluidics, Nguyen and Wereley, Artech House, 2002) and. EP 1 276 555.

Microcarriers may further be encoded, to facilitate their identification. Preferably, the microcarriers of the invention are encoded in such a way that their function, i.e. the type of detection molecule(s) attached to their surface, can be determined by reading the code. The code enables the identification of the microcarrier independently of the performance of the assay. Codes and method for encoding microcarriers are known in the art, and have been disclosed for instance in EP 1 276 555 and EP 1 346 224. Each microcarrier may be encoded, so as to enable identification of single microcarriers within a group of microcarriers. Preferably, microcarriers having the same functionalization harbor the same code, so that the functionalized microcarriers of a set harbor the same code. When several sets of functionalized microcarriers are used, each set is attributed a specific code, in order to distinguish the various sets.

In the cartridge of the invention, the microcarriers are functionalized.

By “functionalized microcarriers” it is herein referred to a microcarrier having detection molecules attached to its surface, that is to say molecules which are capable of binding or reacting with a target molecule or compound. By “reacting with a target molecule” it is herein referred to detection molecules capable of binding specifically with the target molecule. Detection molecules used to functionalize the microcarriers may be proteins, peptides, lipids, sugars and nucleic acids. Preferably, the detection molecule used to functionalize the at least one set of microcarriers is chosen from the list consisting of a protein, a peptide, a DNA fragment, a RNA fragment and a ssDNA fragment, preferably a protein or a peptide. Detection molecules may have any known function. Preferably, detection molecule used to functionalize the at least one set of microcarriers is chosen from the list consisting of an antibody, a receptor, an aptamer and an enzyme.

Preferably, the microcarriers further comprise a label attached to their surface, preferably an activable label. As herein defined, an “activable label” is a label that emits a signal when activated, preferably a light emission. Said label may be a fluorophore or a luminescent molecule, preferably an activable fluorophore or a luminescent molecule. Preferably, the activable label is activated upon binding of the detection molecule to its specific target. Advantageously, the label is a fluorophore-quencher based activable label. Such labels are known in the art and have for instance been described by Ogawa et al. (Mol Pharm.; 6(2): 386-395; 2009).

A “set of functionalized microcarriers” herein refers to one or more microcarriers with the same functionalization, i.e. with the same detection molecule attached to their surface. The set of microcarriers is thus defined at least in part by the detection molecules attached to the microcarriers. In this context, sets of microcarriers are said to be different (i.e. from one another) when at least one detection molecule differs, that is to say when the microcarriers of a set are distinguishable from the microcarriers of the other set by at least one detection molecule attached on the surface of the corresponding microcarriers. A set may be only one microcarrier or a plurality of microcarriers. The microcarriers of one set may carry more than one detection molecules in order to bind or react with two or more target molecules.

Preferably, the microfluidic cartridge comprises more than one set of microcarriers, yet preferably, each of the microchannels of the rnicrofluidic cartridge comprise more than one set of microcarriers. Advantageously, the microfluidic cartridge comprises at least 2, 3, 4, 5, 6, 10, 20 sets of microcarriers, preferably at least 2, 3, 4, 5, 6, 10, 20 different sets of microcarriers. More advantageously, each of the microchannels of the microfluidic cartridge comprise at least 2, 3, 4, 5, 6, 10, 20 sets of microcarriers, preferably at least 2, 3, 4, 5, 6, 10, 20 different sets of microcarriers. In an embodiment, the repartition of the sets of microcarriers may be homogenous in between the microchannels of the rnicrofluidic cartridge, so that all the microchannels comprise the same sets of microcarriers. In another embodiment, each microchannel of the microfluidic cartridge comprises a specific combination of sets of microcarriers.

The microcarriers of the invention are coated with a lyoprotectant. The lyoprotectant enables the preservation of the functionalized microcarriers, and thus the stability of the microfluidic cartridge. The coating of lyoprotectant preferably extends to the internal surface of the microchannel. It should be understood that the microcarriers and the molecules used for their functionalization are thus covered with a coating, i.e. a layer, of lyoprotectant. The lyoprotectant is thus in direct contact with the surface it is intended to cover, that is to say the surface of the microcarriers, and the molecules used for their functionalization.

In the context of the invention, the terms “coated with a lyoprotectant” should be construed as meaning that the microcarriers, and preferably the internal surface of the microchannel, are covered by a layer of lyoprotectant. By “lyoprotectant” it is herein referred to a molecule that protects a biological molecule, such as a protein, a peptide, a lipid, a sugars or a nucleotide, from denaturation and loss of biological activity during dry storage. Lyoprotectants are known in the art and need not be fully listed herein. For instance, many lyoprotectants are polyols, but the class may also include amino acids, peptides, proteins, as well as PHCs, sugars, sugar alcohols, polyvinylpyrrolidones, PEGs, and the like. It should be understood that the definition also includes mixtures of compounds acting as a lyoprotectant, where a first compound and a second compound have a protective effect when used in a mixture.

Preferably, the lyoprotectant is chosen from the list consisting in sugars, sugar alcohols, polyvinylpyrrolidones, amino-acids, proteins and mixtures thereof. More preferably, the lyoprotectant s chosen from the list consisting of sugars and sugar alcohols and mixtures thereof.

According to the invention the term “sugars” herein refers to monosaccharides, disaccharides, trisaccharides and oligosaccharides.

Preferably, the sugar of the invention is chosen from the list consisting of sucrose, trehalose, sorbose, stachyose, gentianose, melezitose, raffinose, fructose, apiose, mannose, maltose, isomahulose, lactose, lactulose, arabinose, xylose, lyxose, digitoxose, fucose, quercitol, allose, altrose, primeverose, ribose, rhamnose, galactose, glyceraldehyde, tagatose, turanose, sophorose, maltotriose, manninotriose, rutinose, scillabiose, cellobiose, gentiobiose, glucose, cellulose and cellulose derivatives, hydroxyethylstarch, soluble starches, dextrans, highly branched, high-mass, hydrophilic polysaccharides. Preferably, cellulose derivatives are chosen from the list consisting of hydroxymethylcellulose, hydroxyethylcellulose, and hydroxypropylmethylcellulose.

The sugar may be a non-reducing or a reducing sugar, preferably a non-reducing sugar. Preferred non-reducing sugars according to the invention are sucrose, trehalose, sorbose, stachyose, gentianose, melezitose and raffinose. Preferred reducing sugars according to the invention are fructose, apiose, mannose, maltose, isomaltulose, lactose, lactulose, arabinose, xylose, lyxose, digitoxose, fucose, quercitol, allose, altrose, primeverose, ribose, rhamnose, galactose, glyceraldehyde, tagatose, turanose, sophorose, maltotriose, manninotriose, rutinose, scillabiose, cellobiose, gentiobiose, and glucose.

According to the invention the term “sugar-alcohol” herein refers to compounds of the general formula HOCH₂(CHOH)ₙCH₂OH. Preferably, the sugar-alcohol according to the invention is chosen from the list consisting of lactitol, mannitol, maltitol, xylitol, crythritol, myoinositol, threitol, sorbitol, and glycerol.

According to the invention the term “amino-acids” when in reference to the lyoprotectant, herein preferably refers to L-amino-acids, preferably L-lysine.

According to the invention the term “protein”, when in reference to the lyoprotectant, is preferably chosen from the list consisting in albumins, preferably bovine serum albumin, gelatins and pectins.

Preservatives are well known in the art as compounds useful to prevent or limit microbial growth or chemical changes, such as oxidation for instance. As such, they are routinely used in compositions comprising biological molecules, such as food., biological samples or cosmetics. They are routinely defined according to their most common uses, and designed as antioxidants, chelating agents, antimicrobial preservatives, or antifungal preservatives.

The inventors have found that these compounds surprisingly enhance the stability of the functionalized microcarriers, preferably when added to the lyoprotectant.

Preferably, the functionalized microcarriers of the invention are further coated with a preservative.

Appropriate antioxidants according to the invention include alpha tocopherol, ascorbic acid, acorbyl palmitate, butylated hydroxyanisole, butylated hydroxytoluene, monothioglycerol, potassium metabisulf[iota]te, propionic acid, propyl gallate, sodium ascorbate, sodium bisulfite, sodium metabisulfite, and sodium sulfite.

Appropriate chelating agents according to the invention include ethylenediaminetetraacetic acid (EDTA), citric acid monohydrate, disodium edetate, dipotassium edetate, edetic acid, fumaric acid, milic acid, phosphoric acid, sodium edetate, tartaric acid, and trisodium edetate.

Appropriate antimicrobial preservatives according to the invention include benzalkoniurn chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, isothiazolinones, in particular methylisothiazolinone, chloromethylisothiazolinone or their mixture, and thimerosal.

Appropriate antifungal preservatives according to the invention include butyl paraben, methyl paraben, ethyl paraben, propyl paraben, benzoic acid, hydroxybenzoic acid, potassium benzoate, potassium sorbate, sodium. benzoate, sodium propionate, and sorbic acid.

It is well known in the art that uncontrolled freeze-thaw of biological molecules, particularly proteins, contributes to their degradation and thus loss of function. Preferably, the microfluidic cartridge of the invention is essentially devoid of any solution, preferably essentially devoid of aqueous solution. In other terms, the microfluidic cartridge preferably does not comprise any solution, i.e. does not comprise any liquid composition, yet preferably does not comprise any aqueous solution, that is to say the microfluidic cartridge is preferably dry. This advantageous embodiment enables the transportation and/or storage of the microfluidic cartridge at any temperature, including temperatures close to or below 0° C., without any risk of damage due to incident freezing.

When in use in a microfluidic device, chemical and/or biological assays are typically performed by flowing fluids in the at least one rnicrochannel of the microfluidic cartridge, while the microcarriers are restricted from exiting said at least one microchannel. The microfluidic cartridge is thus preferably designed to enable the flowing of fluids inside the at least one microchannel without allowing the microcarriers to exit the microchannel.

In an embodiment, the cartridge is packed in a container, preferably a vacuum-sealed container. The container may have any dimension, shape or form appropriate to package the microfluidic cartridge. The container may be made of any material compatible with vacuum sealing. For instance be made of foil, polyethylene (PE), polyvinylidene chloride (PVDC), ethylene vinyl alcohol (EVOH), and/or nylon. The package may be sealed using any conventional technique, such as a thermal press. in another embodiment, the cartridge is packed under a dry gas atmosphere, preferably without oxygen.

In a preferred embodiment, the cartridge is packed in a container which comprises a desiccant. In other terms, in said embodiment, the desiccant is placed in the container with each cartridge. Examples of desiccants which may be useful include silica gel, bentonite, borax, Anhydrone®, magnesium perchlorate, barium oxide, activated alumina, anhydrous calcium chloride, anhydrous calcium sulfate, titanium silicate, anhydrous calcium oxide, and anhydrous magnesium oxide, magnesium sulfate, and Dryrite®, among others, with or without indicator.

The inventors have shown that the cartridge of the invention maintains its properties, i.e. can be used without a loss of accuracy in the detection of the target molecule, even when stored in a wide range of temperature for long periods of time. The microfluidic cartridge of the invention, preferably when packed in a container which comprises a desiccant, is advantageously stable.

By “stable”, it is herein referred to the stability of the sensitivity of detection of the target molecule by the microfluidic cartridge. A cartridge will be considered stable if the sensitivity does not decrease, or decreases of less than 20%. Measurement of the stability may easily be made by comparing the sensitivity, measured as the amount of detection signal measure with the microfluidic device, between a microfluidic cartridge and a reference cartridge (for instance a readily made cartridge), in the same conditions (i.e. using the same sample).

Preferably, the microfluidic cartridge of the invention is stable at a temperature of between −20° C. and 37° C., yet preferably at a temperature of between −20° C. and 37° C. for 2 months, advantageously for 40 days. More preferably, the microfluidic cartridge of the invention is stable at a temperature of between −20° C. and 25° C., yet preferably at a temperature of between −20° C. and 25° C. for 2 months, advantageously for 40 days.

The inventors have further developed a method to appropriately manufacture the microfluidic cartridge of the invention, using usual cartridges pieces such as usual functionalized microcarriers and microchannels. The method includes a step of incubating the functionalized microcarriers, localized with the microchannel of the cartridge, with a stabilizing buffer, for instance a buffer comprising the lyoprotectant, and optionally a preservative. The microchannels are then emptied and dried in conditions that do not compromise the stability of the detection molecules attached to the functionalized microcarriers. Since the behavior of a fluid in a microchannel is different than the behavior of the same fluid in a macrochannel, specific drying condition must be developed in order to avoid destruction and/or alteration of the lyoproectant.

Optionally, the cartridge is packed in a container, preferably in the presence of a desiccant. The resulting cartridge, as already indicated, can conveniently be stored at temperatures ranging from −20° C. to 37° C. during several days, without critical impact on its sensitivity of detection. The cartridge is ready to use and can be transported and stored without the need for refrigeration.

The invention further pertains to a process of manufacture of a micro fluidic cartridge according to the invention, said process comprising:
providing a microfluidic cartridge comprising at least one microchannel and at least a set of functionalized microcarriers, the functionalized microcarriers being localized within the microchannel;
flowing a stabilizing buffer into the at least one microchannel and incubating the functionalized microcarriers with said stabilizing buffer for at least 10 minutes; preferably wherein the stabilizing buffer is a composition comprising a lyoprotectant, yet preferably wherein the lyoprotectant is chosen from the list consisting of sugars and sugar alcohols and mixtures thereof; and
drying the at least one microchannel.

Preferably, the set of functionalized microcarriers, localized within the microchannel, is in suspension in a buffer solution. In practice, during the flowing step, the stabilizing buffer replaces the butler solution.

As just above mentioned, microcarriers are preferably localized within the microchannel in suspension in a buffer solution. This liquid solution containing a suspension of microcarriers is the only form which is available. According to the invention, the buffer solution which is present in the microchannel is replaced by the stabilizing buffer. Consequently, the stabilizing buffer replacing the buffer solution has to allow the deposition of the lyoprotectant on the functionalized microcarriers in spite of potential buffer solution traces on the microcarriers and/or on the microchannel.

Any type of microfluidic cartridge may be used in the process of the invention, provided it comprises at least a microchannel and at least a set of functionalized microcarriers, the functionalized microcarriers being localized within the microchannel, according to the invention. It should be understood that the functionalized microcarriers and microchannels are as those described herein with respect to the cartridge of the invention.

The cartridge of the invention may easily be prepared by the person skilled in the art who will be able to functionalize the microcarriers with molecules of interest, according to the intended use of the cartridge. Methods for attaching a molecule of interest on the surface of a microcarrier are well known in the art.

In the context of the invention, the person skilled in the art may for instance prepare functionalized microcarriers using commercial microcarriers and a molecule of interest, and then introduce the functionalized microcarriers in the at least one microchannel, so as to provide the desired cartridge, to be used in the method of the invention. A classical method for introducing microcarriers into a microchannel is to have them in suspension in a buffer solution which is flown into the microchannel. The buffer used for the introduction of the microchannel does not require specific compounds, and the person skilled in the art may use conventional buffers such as TRIS buffer, HEPES buffer or PBS buffer. Additional interesting methods have been disclosed for instance in WO 00/61198 in WO 04/025560, and in WO 2014/009210. When the process involves more than one set of microcarriers, the sets may be introduced sequentially, to be able to identify the set when the cartridge is used in a microfluidics device. Alternatively, the microcarriers may be encoded, in which case the sets of microcarriers may indifferently be introduced in the microchannel in a random sequence or in a controlled sequence. After the microcarriers have been introduced in the at least one microchannel, in order to facilitate proper arrangement of the microcarriers within the at least one microchannel, the microfluidic cartridge may optionally be tilted or tapped.

Alternatively, the person skilled in the art may introduce unfunctionalized microcarriers in the at least one microchannel, and then proceed with functionalizing the microcarriers while they are already localized in the microchannel. In this case, the unfunctionalized microcarriers are introduced in the at least one microchannel according to methods known in the art, and the microcarriers are further functionalized using methods known in the art, for instance by flowing in the rnicrochannel a solution comprising the molecule of detection to be grafted on the microcarrier, and incubating the microcarriers with said solution.

Once the cartridge according to the invention is available, a stabilizing buffer is flown into the at least one microchannel.

By “stabilizing buffer” it is herein referred to any buffer capable of stabilizing the detection molecules according to the invention. Commercial buffers may be used in this step. Appropriate commercial buffers comprise for instance the buffers WELLChampion commercialized by the company KEm EN TEC Diagnostics, StabilGuard® Immunoassay Stabilizer commercialized by the company SurModics, Liquid Plate Sealer commercialized by the company Candor Bioscience, ELISA Coating Stabilizer commercialized by the company Rockland Immunochemicals, Coating Stabilizer and Blocking Buffer commercialized by the company Meridian Life Science, Immunoassay Blocking Buffer commercialized by the company Abeam ELISA Coating (EC) Stabilizer commercialized by the company Anogen, AppliCoat Plate Stabilizer commercialized by the company AppliChem. Preferably, the stabilizing buffer according to the invention is chosen in the list consisting in Liquid Plate Sealer communercialized by the company Candor Bioscience, Coating Stabilizer and Blocking Buffer commercialized by the company Meridian Life Science, and AppliCoat Plate Stabilizer commercialized by the company AppliChem. Preferably, the stabilizing buffer according to the invention is a composition, preferably a solution, yet preferably an aqueous solution, comprising the lyoprotectant as defined herein. The lyoprotectant is preferably chosen from the list consisting in sugars, sugar alcohols, polyvinylpyrrolidones, amino-acids, proteins and mixtures thereof. More preferably, the lyoprotectant is chosen from the list consisting in sugars and sugar alcohols and mixtures thereof. Advantageously, the stabilizing buffer further comprises at least one preservative as defined herein.

The stabilizing buffer may be flown in the microchannel using a pipet, or using pressure means.

The stabilizing buffer may be flown in the microchannel at room temperature. As classically defined, room temperature is a temperature of between 17 to 25° C. Preferably, the stabilizing buffer is flown in the microchannel at a temperature of about 20° C.

The stabilizing buffer may be flown in the microchannel for more than 30 seconds, preferably for more than 1 minute, yet preferably for around 2 minutes. This enable removing the buffer used to introduce the microcarrier, and ensures that the solution comprised in the microchannel is mostly composed of the stabilizing buffer.

Once the stabilizing buffer has been flown into the at least one microcarrier, the microcarriers are incubated in the presence of said stabilizing buffer for at least 10 minutes, preferably at least 30 minutes, yet preferably at least 1 hour.

The incubation may be performed at room temperature, as defined herein. Preferably, the incubation is performed at 20° C.

After the incubation step, the method comprises a step of drying the at least one microchannel.

In an embodiment, the step of drying the at least one microchannel comprises a step of removing part of the stabilizing buffer from the at least one microchannel.

The stabilizing buffer may be removed by applying positive pressure at one of the extremity of the microchannel, when the microchannel comprises restriction means preferably the entry of the microchannel or possibly the inlet well connected to the entry of the microchannel. Alternatively, it may be possible to apply negative pressure to the outlet of the microchannel so as to suck the stabilizing buffer out of the microchannel.

However, among the different techniques possible for removing the stabilizing buffer, purging the microchannel with a gas seems to be the most efficient. It enables pre-drying of the microchannel, and therefore limits the duration of the drying step.

Preferably, the stabilizing buffer is removed by gas purge, that is to say by flushing said microchannel with a gas under pressure, preferably at one of the extremity of the microchannel, yet preferably the entry when the microchannel comprises restriction means. The gas may be air or any inert gas such as azote. Preferably the gas is used at room temperature as defined herein, yet preferably at about 20° C. Preferably the gas used in dry air, that is to say air having a humidity rate of between 1% and 20%. The gas may be flushed at a positive differential pressure of at least 20 mBar, preferably comprised between 50 mBar and 1500 mBar.

By “positive differential pressure” it is herein referred to the difference of pressure between the pressure used to flush the gas and the pressure in the room or the environment of the microfluidic cartridge. In the context of the invention, this difference is necessarily positive, since the pressure used to flush the gas needs to be superior to that in the room or the environment of the microfluidic cartridge to remove the stabilizing buffer.

When gas purge is used, gas may be flushed in the microchannel during a few seconds to several minutes, preferably between 30 seconds and 15 minutes, more preferably between 10 seconds and 5 minutes.

Too long of an air purge step may compromise the coating of the microcarrier. Similarly, if too much differential pressure is used, the coating may not form properly, thus altering the stability of the microfluidic cartridge. The person skilled in the art may therefore adapt the duration of the air purge according to the differential pressure used, and conversely.

Preferably, the stabilizing buffer is removed from the microchannel by flushing a gas at a positive differential pressure of between 50 mBar and 500 mBar for between 1 and 15 minutes, advantageously for between 1 and 5 minutes, more advantageously for about 3 minutes. Alternatively, the stabilizing buffer is removed from the microchannel by flushing a gas at a positive differential pressure of between 500 mBar and 1500 mBar for between 10 seconds and 1 minute.

Optionally, to facilitate removing the stabilizing buffer, the person skilled in the art may use an absorbing material, positioned outside of the microchannel, at one of its extremity, preferably near or at the outlet and/or the inlet well, so as to absorb the stabilizing buffer in the outlet and/or the inlet well. This is particularly important when the extremities of the microcarrier are connected to inlet and outlet wells. Indeed, fluids tend to accumulate in these wells. In the process of the invention, the step of removing the stabilizing buffer therefore preferably comprises absorbing the flushed stabilizing buffer, preferably with an absorbing material, advantageously positioned at one extremity of the microchannel. The use of such a material increases the flux of the stabilizing buffer toward the outlet, by capillarity. Appropriate absorbing materials in the context of the invention are natural materials such as cotton, linen, hemp, bamboo, silk and synthetic absorbing material. Preferably, the absorbing material is in a solid form, to avoid mixing with the buffer and entering the microchannel. For facility of use, the absorbing material may be used in the form of a random web of fibers, such as for instance wads of cotton, or arranged web of fibers, such as for instance cotton tissue.

Drying of the at least one microchannel may be performed by other techniques, used either instead or in combination with gas purge.

In an embodiment, the step of drying the at least one microchannel is performed by incubating the microfluidic cartridge in a closed chamber, in the presence of dry air. This step is preferably performed using vacuum drying, which fasten drying.

Advantageously, drying the at least one microchannel is performed using vacuum drying, at an absolute pressure comprised between 40 mBar and 700 mBar, preferably between 40 mbar and 60 mBar, yet preferably at an absolute pressure of around 40 mBar. Vacuum drying is preferably performed at room temperature as defined herein, preferably at about 20° C.

Vacuum drying may be performed using any usual appropriate equipment, such as standards vacuum dryers. Such equipment may use desiccant, which are introduced in the chamber to facilitate the drying process. Advantageously, vacuum drying is performed in the presence of a desiccant, preferably as defined herein.

The vacuum drying step may be performed for several hours, preferably for between 1 and 15 hours, However, the drying step, if performed for too long of at an inappropriate pressure, may damage the microfluidic cartridge, or the coating of the microcarrier. The duration of this step is thus better expressed as the dryness to be obtained, that is to say the target humidity rate of the air in the vacuum drying equipment.

Preferably, the at least one microchannel is dried in the conditions detailed herein, until the relative humidity rate of the air inside the vacuum drying equipment reaches between 0.5% and 20%, preferably between 1 and 5%.

By “relative humidity rate”, it is herein referred to ratio of the partial pressure of water vapor to the saturated vapor pressure of water at given pressure and temperature conditions, expressed in percent. In the context of the invention, the “relative humidity rate” is preferably the ratio of the partial pressure of water vapor to the saturated vapor pressure of water at given pressure and temperature conditions.

In an embodiment, the step of drying the at least one microchannel comprises a step of removing part of the stabilizing buffer from the at least one microchannel, preferably using gas purge, followed by a step of incubating the microfluidic cartridge in a closed chamber, in the presence of dry air, advantageously using vacuum drying.

In a preferred embodiment, the step of drying the at least one microchannel comprises:
absorbing the stabilizing buffer in the outlet and/or the inlet well, using an absorbing material, positioned outside of the microchannel, at one of its extremity, preferably near or at the outlet and/or the inlet well;
removing the stabilizing buffer with gas purge, wherein the gas may is flushed in the microchannel during a few seconds to several minutes, preferably between 30 seconds and 15 minutes, more preferably between 10 seconds and 5 minutes; and
drying the at least one microchannel using vacuum drying, advantageously until the humidity rate of the air inside the vacuum drying equipment reaches between 0.5% and 20%, preferably between 1 and 5%, preferably at an absolute pressure comprised between 40 mBar and 700 mBar, preferably between 40 mbar and 60 mBar, yet preferably at an absolute pressure of around 40 mBar.

Optionally, after the step of drying the rnicrochannel, the microfluidic cartridge is packed in a container, preferably a vacuum-sealed container. When the cartridge is packed in a vacuum-sealed container, any appropriate device may be used to create said vacuum. In this case, in order to prevent damage to the microfluidic cartridge, the vacuum inside the vacuum sealed contained should not be different from standard atmosphere of more than 20%. Standard atmosphere is typically defined as a pressure of 101325 Pa (1.01325 bar). The container may have any dimension, shape or form appropriate to package the microfluidic cartridge. The container may be made of any material compatible with vacuum sealing. For instance be made of foil, polyethylene (PE), polyvinylidene chloride (PVDC), ethylene vinyl alcohol (EVOH), and/or nylon. The package may be scaled using any conventional technique, such as a thermal press. In another embodiment, the cartridge is packed under a dry gas atmosphere, preferably without oxygen.

In a preferred embodiment, the cartridge is packed in a container, preferably a vacuum-sealed container, which comprises a desiccant. in other terms, in said embodiment, the desiccant is placed in the container with each cartridge. Examples of desiccants which may be useful include silica gel, bentonite, borax, Anhydrone®, magnesium perchlorate, barium oxide, activated alumina, anhydrous calcium chloride, anhydrous calcium sulfate, titanium silicate, anhydrous calcium oxide, and anhydrous magnesium oxide, magnesium sulfate, and Dryrite®, among others, with or without indicator.

The invention further pertains to the microfluidic cartridge susceptible to be obtained with the process of the invention.

The invention is further described in detail by reference to the following experimental example and the attached figure. This example is provided for purposes of illustration only, and is not intended to be limiting.

### FIGURE LEGEND

FIG. 1: Stability of pre-filed cartridge after storage at different temperatures.

Prefilled cartridges comprising microcarriers functionalized with either an anti-IL-4 antibody (set 1), an anti-II-6 antibody (set 2), an anti-IL-8 antibody (set 3), an anti-TNF-alpha antibody (set 4), an alternative anti-TNF-alpha antibody, different from the set 4 antibody (set 5) or unfunctionalized (set 6) were prepared. They were then stored at −20° C., 4° C., 25° C. or 37° C., and their functionality was tested at different storage time. The functional test consisted in measuring fluorescence in calibrated conditions, and comparing the fluorescence obtained with that obtained with a cartridge of reference.

### EXAMPLES

Prefilled cartridges were prepared as follows:

Empty cartridges comprising microchannels were provided, as well as conventional unfunctionalized microcarriers. Conventional unfunctionalized microcarriers typically harbor chemical moieties (such as streptavidine molecules or carboxyl functions) on their surface so as to enable functionalization with a molecule of interest. Unfunctionalized microcarriers were introduced in the microchannels using conventional techniques. The functionalization of the microcarriers was then performed directly within the microchannels.

Several types of functionalization were tested in separate cartridges. Accordingly:
In a first set of cartridge, the microchannels were flushed with a buffer comprising 1 μg/mL of anti-IL-4 antibody (set 1),
In a second set of cartridge, the microchannels were flushed with a buffer comprising 1 μg/mL of anti-IL-6 antibody (set 2),
In a third set of cartridge, the microchannels were flushed with a buffer comprising 1 μg/mL of anti-IL-8 antibody (set 3),
In a fourth set of cartridge, the microchannels were flushed with a buffer comprising 1 μg/mL of anti-TNF-alpha antibody (set 4),
In a fifth set of cartridge, the microchannels were flushed with a buffer comprising 1 μg/mL of an alternative anti-TNF-alpha antibody, different from the set 4 antibody (set 5).

A sixth set of microcarriers was left unfunctionalized (set 6).

Functionalization of the microcarriers was performed by incubating the microchannels during about 30 minutes to 60 minutes. After incubation, the microchannels were flushed with PBST buffer for one minute, to rinse the antibody. The microchannels were then flushed with stabilizing buffer (Coating Stabilizer and Blocking Buffer commercialized by the company Meridian Life Science). The microchannels and thus the microcarriers were left to incubate in presence of stabilizing buffer for one hour at room temperature.

The microchannels were then dried by:
gas purge, using compressed air at 0.4 bar for 3 minutes.,
followed by evaporation under vacuum for 15 hours at room temperature in a vacuum chamber at around 0.71 bar.

The cartridges were then packed in aluminum bags with silicagel (2 g), under 20% vacuum.

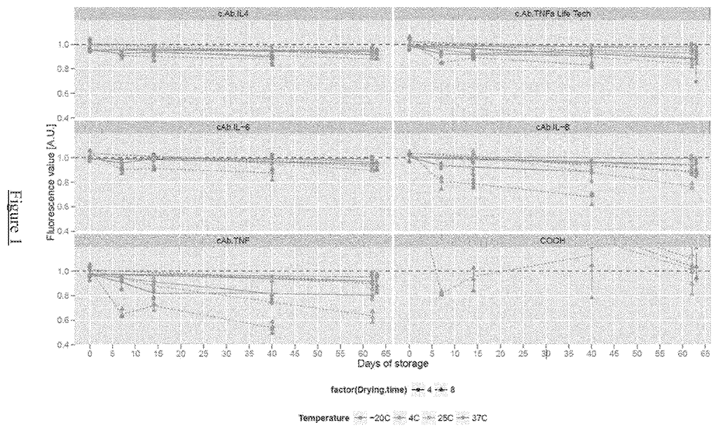

The cartridges were stores at 4° C., 20° C., 25° C. or 37° C., and their functionality was tested at various storage time. The functional test consisted in measuring fluorescence in calibrated conditions, and comparing the fluorescence obtained with that obtained with a cartridge of reference. In each case, the cartridge of reference was a readily made cartridge with microcarriers functionalized with the same antibody as used in the cartridge to be tested. The ratios of fluorescence obtained in different conditions are presented in FIG. 1. The cartridge of the invention can be stored for extended periods of time at any of the temperature tested without any significant loss of functionalization of the microcarriers.

1-19. (canceled)

20. A process of manufacture of a microfluidic cartridge, the micro fluidic cartridge comprising: at least one microchannel, wherein the microchannel is a hollow structure configured for the passage of fluids; and at least a set of functionalized microcarriers, wherein the microcarriers are localized within the microchannel, and wherein the functionalized microcarriers are coated with at least a lyoprotectant;
said process comprising:
providing a microfluidic cartridge comprising at least one microchannel and at least a set of functionalized microcarriers in suspension in a buffer solution, the microcarriers being localized within the microchannel;
flowing a stabilizing buffer into the at least one microchannel and incubating the functionalized microcarriers with said stabilizing buffer for at least 10 minutes, wherein the stabilizing buffer is a composition comprising the lyoprotectant; and
drying the at least one microchannel.

21. The process of claim 20, wherein the lyoprotectant is or comprises a sugar selected from the list consisting of sucrose, trehalose, sorbose, stachyose, gentianose, melezitose, raffinose, fructose, apiose, mannose, maltose, isomaltulose, lactose, lactulose, arabinose, xylose, lyxose, digitoxose, fucose, quercitol, allose, altrose, primeverose, ribose, rhamnose, galactose, glyceraldehyde, tagatose, turanose, sophorose, maltotriose, manninotriose, rutinose, scillabiose, cellobiose, gentiobiose, glucose, cellulose and cellulose derivatives, hydroxyethylstarch, soluble starches, dextrans, highly branched, high-mass, hydrophilic polysaccharides.

22. The process of claim 20, wherein the lyoprotectant is or comprises a sugar-alcohol selected from the list consisting of lactitol, mannitol, maltitol, xylitol, erythritol, myoinositol, threitol, sorbitol, and glycerol.

23. The process of claim 20, wherein the stabilizing buffer is flown in the at least one microchannel at room temperature for more than 30 seconds.

24. The process of claim 20, wherein the microcarriers are incubated in the presence of said stabilizing buffer at room temperature for at least 10 minutes.

25. The process of claim 20, wherein the step of drying the at least one microchannel comprises a step of removing part of the stabilizing buffer from the at least one microchannel by flushing said microchannel with a gas under pressure.

26. The process of claim 20, wherein the step of drying the at least one microchannel comprises a step of removing part of the stabilizing buffer from the at least one microchannel by vacuum drying.

27. The process of claim 20, wherein the step of drying the at least one microchannel comprises absorbing the stabilizing buffer.

28. The process of claim 25, wherein the step of removing part of the stabilizing buffer from the at least one microchannel by flushing said microchannel with a gas under pressure is followed by a step of incubating the microfluidic cartridge in a closed chamber, in the presence of dry air.

29. The process of claim 25, wherein the step of drying the at least one microchannel comprises removing the stabilizing buffer by flushing said microchannel with a gas under a positive differential pressure of at least 20 mBar.

30. The process of claim 26, wherein the step of drying the at least one microchannel comprises a step of vacuum drying at an absolute pressure between 40 mBar and 700 mBar.

31. The process of claim 30, wherein the at least one microchannel is dried until the humidity rate of the air inside the vacuum drying equipment reaches between 0.5% and 20%.

32. The process of claim 27, wherein absorbing the stabilizing buffer comprises use of an absorbing material positioned at one extremity of the microchannel.

33. The process of claim 20, further comprising a step of packing the microfluidic cartridge in a container.

34. The process of claim 33, wherein the container is vacuum sealed after the microfluidic cartridge is packed in the container.

35. The process of claim 33, wherein the container comprises a desiccant.

36. The process of claim 20, wherein the surface of the microcarriers and the internal surface of the microchannel are coated with said lyoprotectant.

37. The process of claim 20, wherein the at least one set of microcarriers are functionalized with a detection molecule, wherein said detection molecule is selected from the list consisting of a protein, a peptide, a DNA fragment, a RNA fragment and a ssDNA fragment.

38. The process of claim 20, wherein the microfluidic cartridge comprises more than one microchannel, and wherein each of the microchannels of the microfluidic cartridge comprises at least 2, 3, 4, 5, 6, 10, 20 sets of microcarriers.

39. A microfluidic cartridge produced by the process according to claim 20.
